(19) United States
(12) Patent Application Publication
(10) Pub. No.: US 2022/0378440 A1
(43) Pub. Date: Dec. 1, 2022

(54) MOTOR CONTROL SYSTEM FOR DYNAMICALLY SWITCHING SHAVER MOTOR CONTROL PROTOCOLS

(71) Applicant: ARTHREX, INC., Naples, FL (US)

(72) Inventors: Robert Fugerer, Lutz, FL (US); Robert Breckner, Reno, NV (US)

(73) Assignee: ARTHREX, INC., Naples, FL (US)

(21) Appl. No.: 17/329,941

(22) Filed: May 25, 2021

(51) Int. Cl. A61B 17/16; A61B 17/17

(52) U.S. Cl. CPC A61B 17/1628; A61B 17/1615; A61B 17/1707; A61B 17/1624; A61B 2017/00039

(57) ABSTRACT

A medical device system configured to dynamically switch motor control protocols while a motor within a handheld device is operating to increase efficiency of the motor operation and to provide improved reliability and performance is disclosed. In at least one embodiment, the medical device system may be configured to dynamically switch motor control protocols while the motor is operating based on input from one or more sensors configured to monitor a motor, including, but not limited to, monitoring a magnetic flux field of the motor or monitoring current to the motor. The medical device system may dynamically switch motor control protocols between motor control protocols, including, but not limited to, Six-Step Commutation, Hall-Based Sinusoidal Commutation and Field Oriented Commutation.

### BACKGROUND

The disclosure relates generally to handheld, rotary medical devices, and more particularly, to motor control systems for driving handheld, rotary medical devices with shavers.

Handheld rotary medical devices include working ends, which are often shavers or burrs that are configured for the removal of hard or soft tissue from the body. Many of these devices are configured to remove soft tissue. The general console requirements for power and shaver tools include forward and reverse motor rotation at speeds from as low as a few revolutions per minute (RPM) to as high as 75,000 RPM over a wide range of torque profiles, depending on the desired tool function and gear ratio. In many cases, a motor is required to start smoothly under high loads while maintaining low speed at high torque, while at other times a motor needs to consistently accelerate to a high RPM while monitoring dynamic load changes to limit over-current conditions.

Current methods for controlling shaver motors is limited to a six-step commutation approach. As such, these current systems are limited to the advantages and disadvantages of the six-step commutation approach regardless of the application and the requirements of such application. Different surgeries have different requirements, such as working on the various tissues, cartilage and bone found in the human body. The numerous types of tissue in patients are not suited to a one size fits all approach. Rather, each material has different requirements of shaver operation for the material to be best handled.

### SUMMARY

A medical device system configured to dynamically switch motor control protocols while a motor within a handheld device is operating to increase efficiency of the motor operation and to provide improved reliability and performance is disclosed. In at least one embodiment, the medical device system may be configured to dynamically switch motor control protocols while the motor is operating. A controller within the medical device system monitors input from one or more sensors configured to monitor a magnetic flux field of the motor or input from one or more sensors configured to monitor current to the motor. In at least one embodiment, the controller may dynamically switch motor control protocols between motor control protocols, including, but not limited to, Six-Step Commutation, Hall-Based Sinusoidal Commutation and Field Oriented Commutation, based on the input from the one or more sensors.

In at least one embodiment, the medical device system may include a handheld rotary medical device formed from a motor, an inner drive shaft coupled to the motor, one or more sensors configured to sense a magnetic flux field of the motor, an elongated, tubular, outer housing encapsulating the inner drive shaft such that the inner drive shaft is positioned within the outer housing and a working element at a distal end of the inner drive shaft. The medical device system may include one or more sensors configured to monitor the magnetic flux field of the motor or one or more sensors configured to sense current to the motor, or both. The medical device system may include a memory that stores instructions and a processor that executes the instructions to perform operations. The operations may include controlling the driving of the motor and the inner drive shaft, wherein the processor is configured to monitor the sensor configured to monitor the magnetic flux field of the motor and the sensor configured to monitor current to the motor. The operations may include dynamically switching motor control protocols while the motor is operating based on input from the sensor configured to monitor the magnetic flux field of the motor or input from the sensor configured to monitor current to the motor, or both.

The processor may be configured to perform operations to operate the motor via one or more of the following motor control protocols at different times: Six-Step Commutation, Hall-Based Sinusoidal Commutation, Field Oriented Commutation, other motor control methods and motor control protocols yet to be conceived. In at least one embodiment, the motor may be a brushless direct current motor. The processor may perform operations based on instructions to dynamically switch motor control protocols while the motor is operating based on input from the sensor configured to monitor the magnetic flux field of the motor or based on input from the sensor configured to monitor current to the motor, or both. In at least one embodiment, the sensor configured to sense a magnetic flux field of the motor may be one or more hall sensors.

In at least one embodiment, the processor may automatically detect a presence of the sensor configured to sense a magnetic flux field of the motor, such as, but not limited to one or more hall sensors on the motor. The processor may also be configured to drive the motor continuously upon detection of failure of a component of the system. In at least one embodiment, the failed component may be one or both of the sensors, one sensor being configured to monitor the magnetic flux field of the motor and the other sensor configured to configured to monitor current to the motor. The system may be configured to enable a user, upon being alerted of a sensor failure, to have the option to either seek a replacement handpiece to continue a surgical procedure or to have the system operate the handpiece in a less efficient motor control protocol not needing the failed sensor but nonetheless enabling the surgeon to complete the surgical procedure using the same handpiece without the delay of seeking a replacement device.

The dynamically switched motor control protocols may include protocols based on results from comparing calculated parameters from the sensors against thresholds. In at least one embodiment, the thresholds may be fixed. In another embodiment, the thresholds may be a function of measured noise floors which the processor uses to calculate minimum allowable signal to noise thresholds.

An advantage of the system is that the system can operate as an automatic, on-the-fly system that switches between the different motor control methods with the intent of optimizing the overall control of the shaver tools and hand-pieces.

Another advantage of the system is that the system may switch between different motor control methods while the motor is operating.

Yet another advantage of the system is that the system is configured to monitor the health of the motor so that if there is a detectable failure in the handpiece operation, the procedure could continue to proceed without causing a stop in the workflow. This may be advantageous in surgical procedures where upon detecting, for example, a sensor failure, it is more desirable to complete the procedure using what may be a suboptimal sensorless control method for that procedure, as opposed to causing a disruption in the surgical operation. Continued use of the handpiece in a suboptimal condition would be the surgeon's decision to make, and appropriate messaging to replace or reset the handpiece would be passed up through the proper system channels to the surgeon.

Another advantage of the system is that the system integrates hall based and back electromotive forces (BEMF) brushless (BLDC) motor commutation protocols with use of an augmented hall based motor commutation protocol that is sinusoidal, reducing operation noise and vibration, as well as a field oriented control motor protocol, which is also sensorless and has still lower noise and vibration characteristics.

Still another advantage of the system is that the system may dynamically switch between multiple motor control protocols to optimize specific operational performance and take advantage of the strengths and weaknesses of the different control methods by automatically detecting when one motor control protocol should be used over the other and switching between them dynamically in real-time while controlling the motor.

Another advantage of the system is that oscillation modes of the handpiece can be optimized using the system.

Yet another advantage of the system is that the system supports a mix of low-speed, torque, thermal and electrical efficiencies in handheld devices that heretofore hasn't been possible thereby enabling new handheld devices to be developed that would have such requirements.

Another advantage of the system is that system extends the usable life of the motor.

Still another advantage of the system is that the system is operable over a wide range of speed-torque ranges, position tracking and efficiency requirements.

Another advantage of the system is that this system functions to control motor parameters to keep the parameters from falling out of acceptable ranges.

Yet another advantage of the system is that the processor functions to maintain a motor run-time vector (position, speed, acceleration and stability) throughout operation of the motor.

Another advantage of the system is that the processor choses motor control protocols based on torque, signal-to-noise ratios and run-time vector thresholds.

These and other embodiments are described in more detail below.

### BRIEF DESCRIPTION OF THE DRAWINGS

FIG. 1 is a perspective view of an exemplary handheld rotary medical device with a shaver as a working element of the medical device system and configured to be operated according to the motor control protocols set forth in FIGS. 5-8.

FIG. 2 is an exploded side of the shaver shown in FIG. 1.

FIG. 3 is an assembled side view of the shaver shown in FIG. 2.

FIG. 4 is cross-sectional view of the shaver taken as section line 4-4 in FIG. 3.

FIG. 5 is a schematic diagram of implementation of dynamically switching motor control protocols of the medical device system, while a motor within a handheld device is operating, to increase efficiency of the motor operation and to provide improved reliability and performance.

FIG. 6 is a schematic diagram of an automatic selection of the dynamically switching motor control protocols of the medical device system by the processor.

FIG. 7 is a schematic diagram of a manual selection of the dynamically switching motor control protocols of the medical device system by the user.

FIG. 8 represents data taken from a console controller of the system where the motor winding currents were sampled in real time while switching between various modes of motor control protocols including a six step motor control protocol, a hall based sinusoidal motor control protocol and a field oriented motor control protocol while the motor was running.

FIG. 9 is a graph of common shaver handpiece functionality related to starting torque & speed of the system.

### DETAILED DESCRIPTION

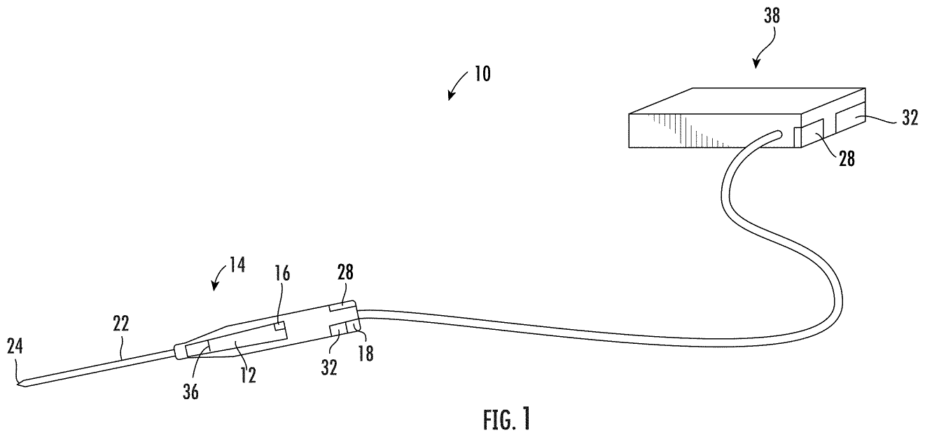

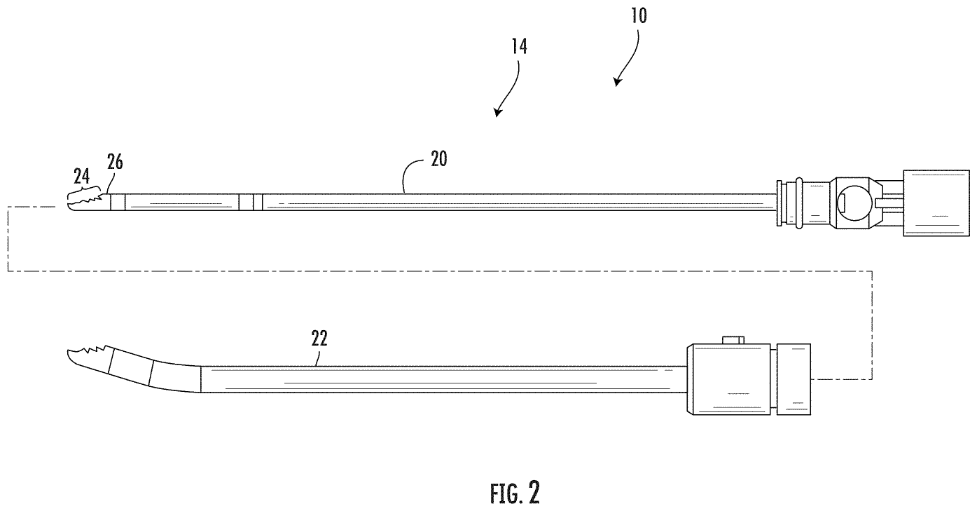

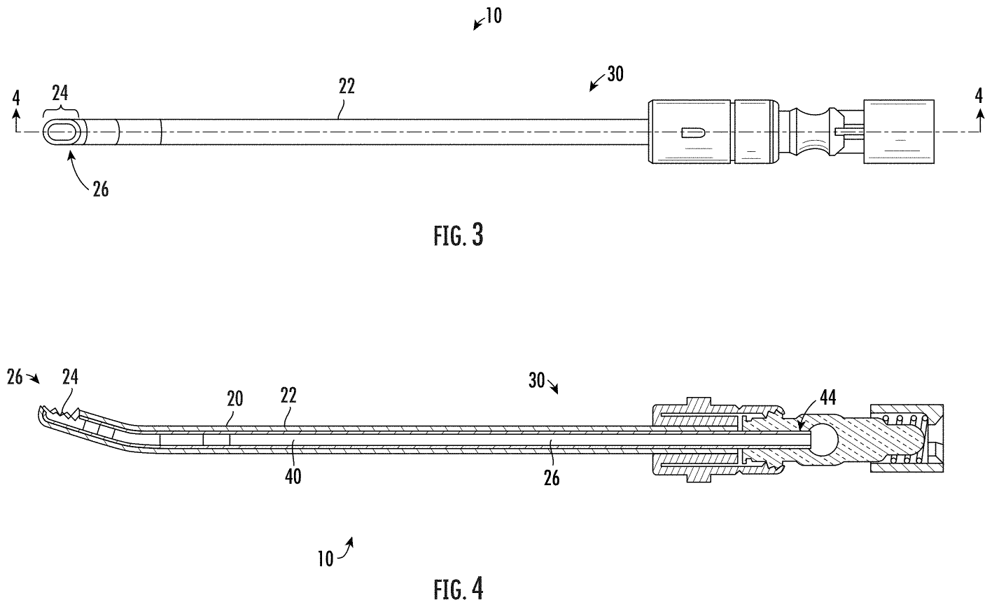

As shown in FIGS. 1-9, a medical device system 10 configured to dynamically switch motor control protocols while a motor 12 within a handheld device 14 is operating to increase efficiency of the motor operation and to provide improved reliability and performance is disclosed. In at least one embodiment, the medical device system 10 may be configured to dynamically switch motor control protocols while the motor 12 is operating based on input from one or more sensors 16 configured to monitor a magnetic flux field of the motor 12 or input from one or more sensors 18 configured to monitor current to the motor 12, or both. In at least one embodiment, the medical device system 10 may dynamically switch motor control protocols between motor control protocols, including, but not limited to, Six-Step Commutation 60, Hall-Based Sinusoidal Commutation 64, Field Oriented Commutation 62, shown in FIG. 8, other motor control protocols and motor control protocols yet to be conceived.

In at least one embodiment, as shown in FIGS. 1-4, the medical device system 10 may include a handheld rotary medical device 14 that may include a motor 12 and an inner drive shaft 20 coupled to the motor 12. In at least one embodiment, the motor 12 may be, but is not limited to being, a brushless direct current motor (BLDC). The medical device system 10 may include one or more sensors 16 configured to sense a magnetic flux field of the motor 12. The sensor 16 may be, but is not limited to being, one or more hall sensors, analog-to-digital converters and other devices. For example, the sensor 16 may be one or more hall sensors mounted to the motor shaft 36 of the motor 12 that provides rotational position information back to a processor 32 in a controller 38, which may also be referred to as a main console. The sensor 16 may also be one or more hall sensors mounted on a gearbox shaft of a motor 12 that provides positional information to processor 32 directly or through an encoder that is configured to monitor position with high resolution while also providing that information to processor 32 over a plurality of digital protocols such as I2C, SPI, CAN and the like. The sensor 16 may also be one or more hall sensors mounted on a gearbox shaft of a motor 12 that provides positional information to processor 32, together with the gear ratio of the gearbox shaft of the motor 12.

The sensor 16 may also be one or more analog-to-digital converters that sense current and voltage of the drive signals of the motor 12 and then use the calculations in the processor 32 to measure back-emf voltage, which may be used to identify position, and to measure current, which may be used to identify the amount of torque present. In at least one embodiment, the sensor 16 may be one or more analog-to-digital converters positioned within the controller 38. The sensor 16 may also be an analog-to-digital converter used for noise level calculations of the drive current of the motor 12 and back electromotive force (BEMF) to identify a signal-to-noise ratio threshold for accurately identifying motor position and torque.

The handheld rotary medical device 14, as shown in FIGS. 1-4, may include an elongated, tubular, outer housing 22 encapsulating the inner drive shaft 20 such that the inner drive shaft 20 is positioned within the outer housing 22. The handheld rotary medical device 14 may include a working element 24 at a distal end 26 of the inner drive shaft 20. In at least one embodiment, the working element 24 may be, but is not limited to being, a shaver. The handheld rotary medical device 14 may include one or more sensors 18, as shown in FIG. 1, configured to sense current to the motor 12. The sensor 18 may be, but is not limited to being, one or more of the sensors previously identified herein in connection with sensor 16. In at least one embodiment, the sensor 18 configured to sense current to the motor 12 may be configured to sense back electromotive force (BEMF) generated by the motor magnets. The medical device system 10 may be configured such that when the device 14 is attached to the system 10 or when an attachment is secured to the handpiece, the processor 32 may identify operational parameters, such as, but not limited to, speed and torque, associated with that device, which may be stored within the memory, on the device itself or elsewhere. Those parameters in storage may be communicated to the processor 32 via wired or wireless communication systems.

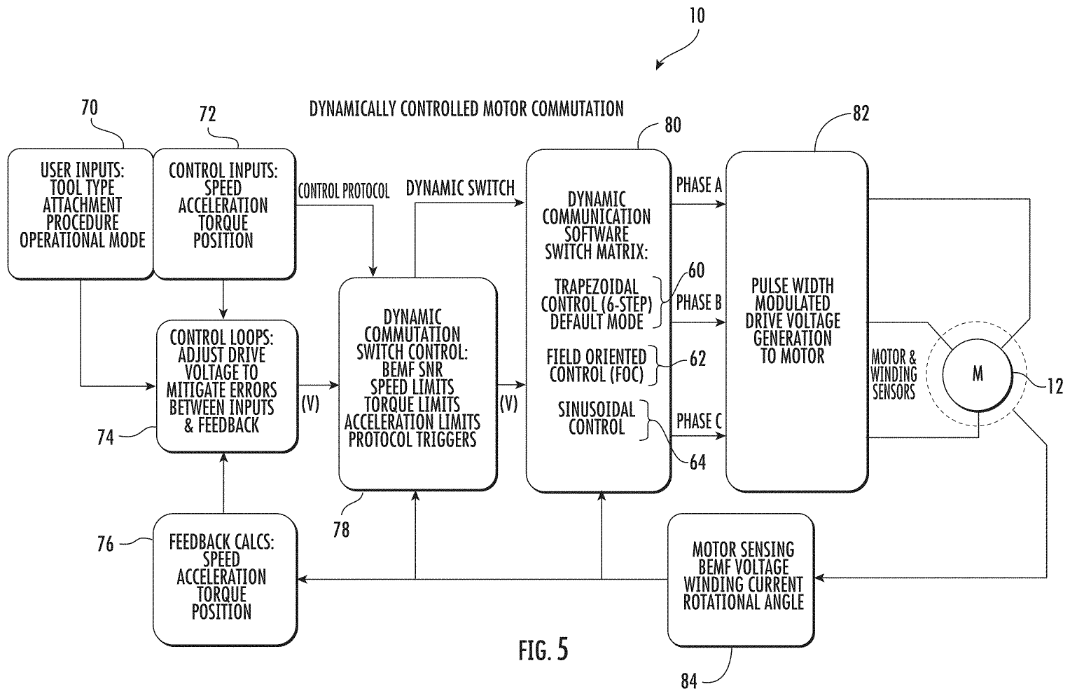

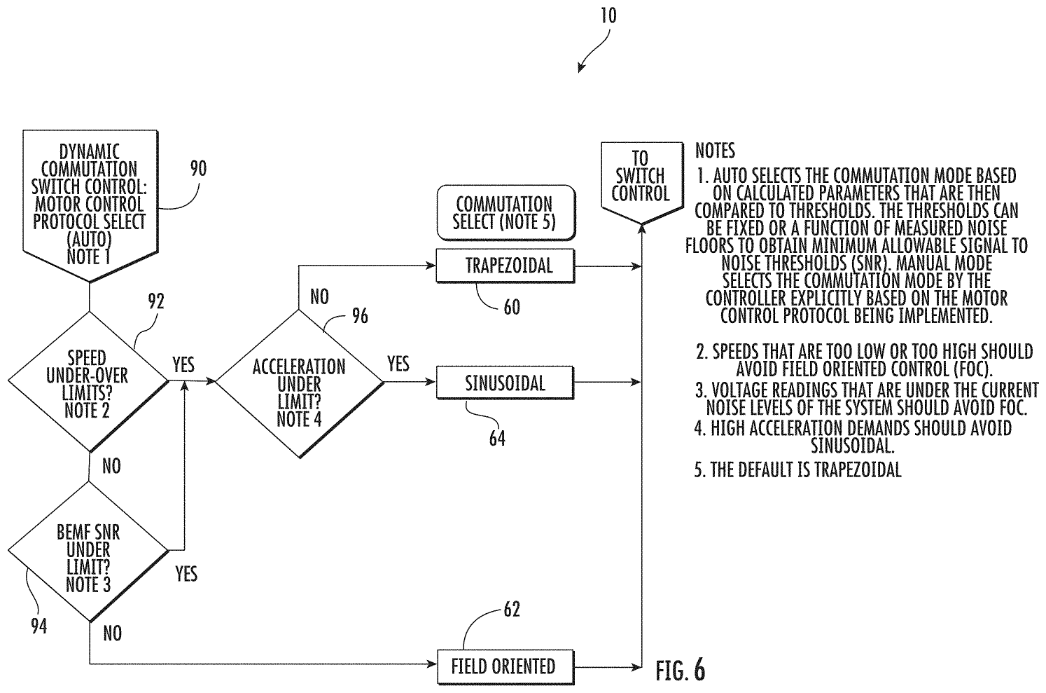

The medical device system 10 may include memory 28, as shown in FIG. 1, that stores instructions and a processor 32 that executes the instructions to perform operations. The operations, as shown in FIGS. 5-7, may include controlling the driving of the motor 12 and the inner drive shaft 20, wherein the processor 32 is configured to monitor the sensor 16 that monitors the magnetic flux field of the motor 12 or the sensor 18 that monitors current to the motor 12, or both. The operations may also include dynamically switching motor control protocols while the motor 12 is operating based on input from the sensor 16 configured to monitor the magnetic flux field of the motor 12 or input from the sensor 18 configured to monitor current to the motor 12, or both. The processor 32 that performs operations based on instructions to dynamically switch motor control protocols may be configured to perform operations to operate the motor 12 via at least one of the following motor control protocols at different times, including, but not limited to, a Six-Step Commutation 60, a Hall-Based Sinusoidal Commutation 64, a Field Oriented Commutation 62 other motor control protocols and motor control protocols yet to be conceived. In at least one embodiment, the processor 32 may dynamically switch motor control protocols between two of Six-Step Commutation 60, Hall-Based Sinusoidal Commutation 64 and Field Oriented Commutation 62 based on the input received.

The processor 32 may dynamically switch between all three methods to optimize specific operational performance, as shown in FIGS. 5-8. By dynamically switching between all three methods, this approach takes advantage of the strengths and weaknesses of the different control methods, by automatically detecting when one motor control protocol should be used over the others and switching between them dynamically in real-time while controlling the motor 12. The processor 32 may perform operations based on instructions to dynamically switch motor control protocols while the motor 12 is operating based on input from one or more sensors configured to monitor the motor 12. In at least one embodiment, the processor 32 may perform operations based on instructions to dynamically switch motor control protocols while the motor 12 is operating based on input from the sensor 16 configured to monitor the magnetic flux field of the motor 12 or input from the sensor 18 configured to monitor current to the motor 12, or both. In at least one embodiment, the sensor 16 configured to sense a magnetic flux field of the motor 12 is at least one hall sensor. The sensor 16 may be one or a plurality of sensors 16 configured to monitor the magnetic flux field of the motor 12. In at least one embodiment, the system 10 may include at least three sensors 16 configured to monitor the magnetic flux field of the motor 12.

When hall sensors are present, the system 10 can use BLDC motor control protocols. This will create scenarios that allow the best algorithm to be implemented automatically, while also maintaining the positional accuracy that is needed for window lock functionality of the working element 24. Speed may be maintained with a proportional, integral, derivative control loop (PID) that compares set speed to measured speed and adjusts motor current through a pulse width modulated (PWM) signal that sends more or less voltage to the motor. As load increases on the motor 12, the speed drops and additional current is needed, which is supplied by a higher PWM duty cycle to the console voltages driving the three phases of the brushless direct current (BLDC) motor. Additionally, even when the hall sensors are present, the system 10 can take advantage of the strengths and weaknesses of the different motor control protocol by automatically determining when one motor control protocol should be used over the others and switching between them dynamically in real-time while controlling the motor 12.

In at least one embodiment, as the motor 12 rotates, multiple hall sensors 16, such as, but not limited to, three hall sensors located on the motor 12, may sense changes in the motor's magnetic flux field and signal the position of the motor shaft 36 to the controller 38, which allows the processor 32 to accurately sequence the voltage drivers thereby enabling the motor shaft 36 to accurately sequence the motor 12 turning in a consistent direction. By reversing the sequence, the rotation can be changed from forward to reverse. In at least one embodiment, the processor 32 may automatically detect the presence of the one or more sensors 16 configured to sense a magnetic flux field of the motor 12.

As shown in FIG. 5, a user may provide user input 70 to the system 10 via any appropriate manner, such as, but not limited to the console 38, as shown in FIG. 1, an interface, such as, but not limited to, a screen, monitor, graphical user interface, and the like which may or may not be incorporated within a computer, a laptop, a tablet device, a phablet, a server, a mobile device, a smartphone, a smart watch, and/or any other type of computing device. The system 10 may receive user input 70 such as, but not limited to, a type of tool attachment attached to the system 10, operational specifications associated with the tool attachment, such as, but not limited to, speed, torque and the like, type of procedure to be undertaken by a surgeon with the handheld device 14 of the system 10, the operational mode of the handheld device 14, such as, but not limited to, oscillation mode, continuous mode, high speed mode, moderate speed mode, low speed mode, high torque mode, low torque mode, and the like. The processor 32 may receive this input, and together with the information residing within the memory 28, may control the handheld device 14 as set forth herein.

The system 10, as shown in FIG. 5, may receive control input 72 including, but not limited to, rotational speed of the working element 24, acceleration, torque, rotational position of the working element 24 and the like. The processor 32 may control the motor with control loops 74 to adjust drive voltage to mitigate errors between inputs and feedback. The processor 32 may perform feedback calculations 76 to control the motor 12 with speed, acceleration, torque and position. The processor 32 may be configured for dynamic commutation switch control factors 78 of the motor control protocol based on back electromagnetic force (BEMF) signal-to-noise ratio (SNR), speed limits, torque limits, acceleration limits, motor protocol triggers and the like. The processor 32 may be configured to dynamically switch the motor operating protocol while the motor 12 is operating or when the motor 12 is stopped. The dynamic commutation protocol switch matrix 80 enables the processor 32 to switch between a trapezoidal (6-Step) motor control protocol 60, a field oriented motor control protocol (FOC) 62, sinusoidal motor control protocol 64, other motor control protocols and motor control protocols yet to be conceived. In at least one embodiment, the trapezoidal (6-Step) motor control protocol 60 may be the default motor protocol mode. Once a motor control protocol has been selected, the processor 32 may drive the motor 12 according to the selected motor control protocol via pulse width modulated drive voltage at 82. The processor 32 may receive signals from the sensors 16,18 at 84 to perform the feedback calculations set forth herein.

In at least one embodiment, as shown in FIG. 6, the system 10 may be configured such that the processor 32 executes instructions to perform operations in which the processer 32 dynamically switch motor control protocols at 90 based on results from comparing calculated parameters from the sensors 16, 18 as compared against thresholds. In at least one embodiment, the thresholds may be fixed. The thresholds may also be a function of measured noise floors which the processor 32 uses to calculate minimum allowable signal to noise thresholds. The system 10 may be placed in manual or automatic modes via input from a user, such as with the handpiece 14 or other input device. When the system 10 is placed in automatic mode, as shown in FIG. 6, the processor 32 selects the commutation mode based on calculated parameters that are then compared to thresholds. The thresholds can be fixed or a function of measured noise floors to obtain minimum allowable signal to noise ratio (SNR) thresholds.

The processor 32 may operate to implement a motor control protocol that best fits the situation at hand. At times, the processor 32 may be able to implement a motor control protocol that is the most efficient under the circumstances. Thus, the system 10 may dynamically switch between all of the motor control protocols to optimize specific operational performance. Each of the motor control protocols has advantages that are not found in the other motor control methods. In other times, such as when a sensor has malfunctioned, the processor 32 implements a motor control protocol that enables the device 14 to continue to be used even though the motor control protocol implemented by the processor 32 may not be the most efficient if all components of the system 10 were operable. In determining which motor control protocol the processor 32 is to implement, as shown in FIG. 6, the processor 32 executes operations based on the following instructions. The processor determines whether the speeds are over or under limits at 92 and, in general, the processor 32 may avoid using the Field Oriented Control (FOC) motor control protocol 62 to operate the handheld device 14 at speeds that are too low or too high. The speed ranges may be determined by system considerations that may vary based on a specific motor implements in the system 10. For example, high gear ratios would enable FOC 62 to be used at lower speeds than a motor 12 at lower gear ratios but would also limit maximum speed. The processor 32 may monitor signal-to-noise at the low end of the speed spectrum because low speeds make it difficult to measure a back electromagnetic force. Computational limits of the processor 32 may limit the high end of the motor speed spectrum because the processor 32 cannot process required computations required to maintain the high speeds. The operational speed limits can be precalculated for the system 10 by first benchmarking the time required to make the calculations and then based on desired speed, gear ratio and motor inductance, establishing an upper limit for FOC.

If the processor 32 determines that the handheld device 14 is to be driven at speeds that are not too low or too high at 92, then the processor whether BEMF SMR is under a threshold at 94. If the handheld device 14 is not under a threshold at 94, then the processor selects a field oriented motor control protocol 62. The processor 32 avoids using the Field Oriented Control motor control protocol 62 if voltage readings are under the current noise levels of the system.

If the processor 32 determines that the handheld device 14 is over or under a speed threshold at 92 or is under a BEMF SNR threshold at 94, then the processor 32 determines at 96 whether acceleration is under a threshold. The processor 32 may avoid using the Sinusoidal motor control protocol 64 if the desired usage of the handheld device 14 calls for high acceleration demands. If the desired usage of the handheld device 14 is under an acceleration limit, then the processor 32 uses the sinusoidal motor control protocol 64. If the desired usage of the handheld device 14 is over an acceleration limit, then the processor 32 uses the trapezoidal motor control protocol 60. In at least one embodiment, the processor 32 may use the Trapezoidal motor control protocol as the default motor control protocol.

Use of motor control protocols which use BEMF sensors is advantageous when starting the motor 12 or when operating the motor 12 at lower speeds. Such is the case because low motor speeds generate low currents making motor control via current sensors difficult and the signal-to-noise ratio required to accurately detect BEMF with current sensors becomes a factor. Hall sensors 16 do not suffer from such issues because the sensors 16 are detecting the magnetic field of the magnets in the BLDC motor, which decouples detection of the position of the motor 12 from the speed of the motor 12.

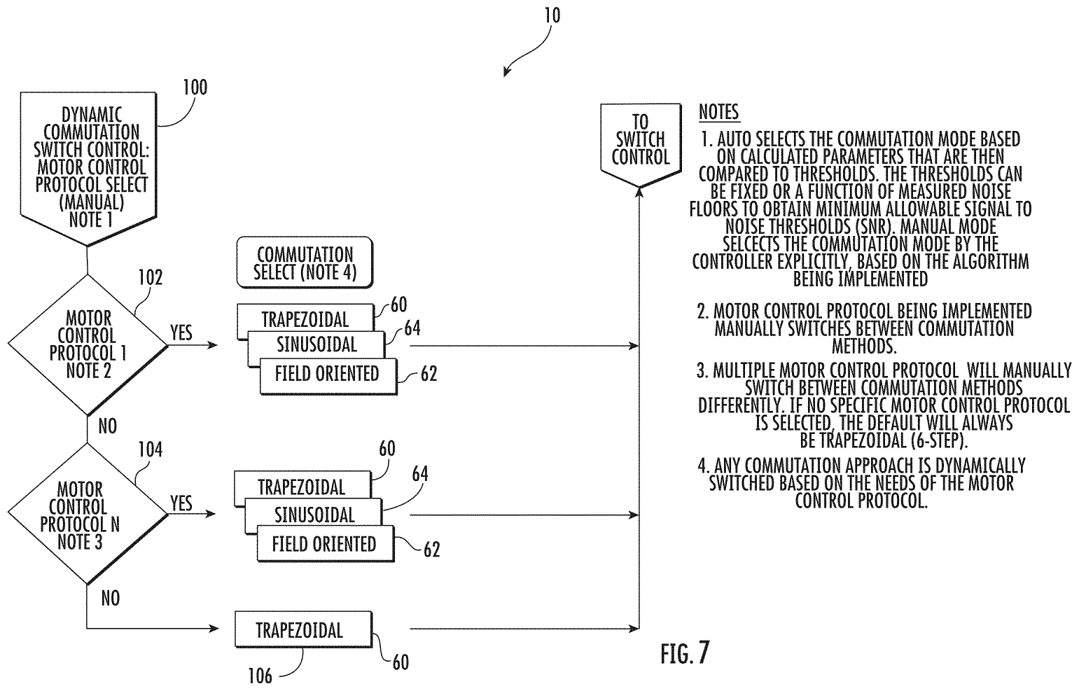

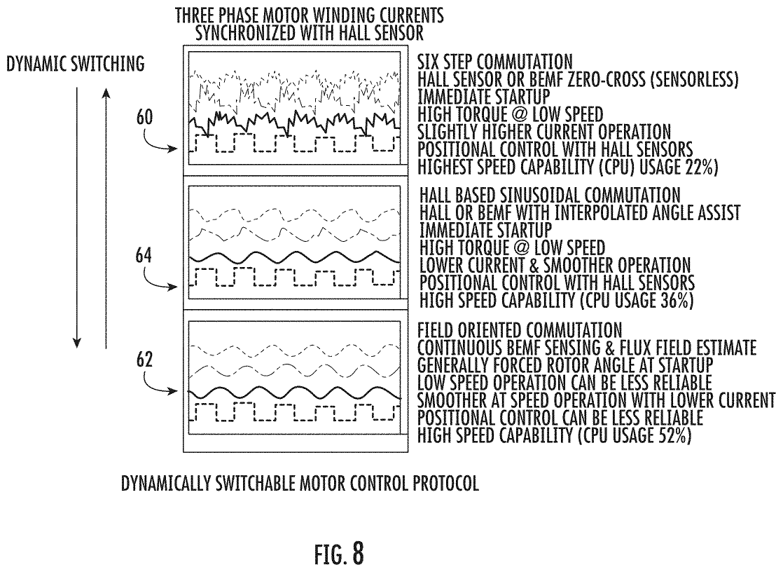

When the system 10 is in manual mode, as shown in FIG. 7, the processor 32 selects the commutation mode by the controller 38 explicitly based on data entered by a user at 100, such as, but not limited to, a surgeon or other medical professional, as to the type of surgical application in which the handpiece is to be used. In the manual mode, a user input overrides the automatic mode and operates based off of the input from the user. In the manual mode, a user, such as, but not limited to, a surgeon or other medical professional, may enter a tool type, such as a drill, and the like, a type of attachment in the tool, operational conditions, such as speed, torque, and the like, a mode of operation, such as oscillation, continuous and the like, and other conditions, and optionally, a desired motor control protocol. Thus, when in manual mode, a user may or may not input a desired motor control protocol. In at least one embodiment, a user designates which motor control protocol is to be used at 102 to operate the handheld device 14. The controller 38 may select the parameters of operating the handpiece once the type of surgical application has been entered into the system 10. In at least one embodiment, a user may have a profile stored within the memory 28 of the system 10. The profile may include parameters particular to that user which may include, but is not limited to including, operational speeds, torques for different tools, surgical procedures, and other options.

The processor 32 choses at 104 a motor control protocol 60, 62, 64 that best matches the input from a user. If a match cannot be made and no motor control protocol is selected, then the processor 32 choses the default motor control protocol at 106, which is the trapezoidal motor control protocol 60. As previously set forth, any commutation approach is dynamically switched by the processor 32 based on the needs of the motor control protocol.

As shown in FIG. 8, the motor 12 may be controlled with one of a number of motor control protocols. In at least one embodiment, the motor 12 may be controlled via three phase motor winding currents synchronized with one or more hall sensors 16. The six-step commutation motor control protocol 60 may be implemented with one or more hall sensors 16 or a BEMF zero-cross sensorless configuration. The six-step commutation motor control protocol 60 provides immediate motor start-up and high torque at low speed with slightly higher current draw during motor operation. Positional control of the working element 24 may be maintained via one or more hall sensors 16.

The sinusoidal commutation motor control protocol 64 shown in FIG. 8 may be implemented with one or more hall sensors 16 or BEMF with interpolated angle assist. The sinusoidal commutation motor control protocol 64 provides immediate motor start-up and high torque at low speed with slightly lower current draw and smooth motor operation. Positional control of the working element 24 may be maintained via one or more hall sensors 16.

The field oriented commutation motor control protocol 62 shown in FIG. 8 may be implemented with continuous BEMF sensing and flux field estimate. The field oriented commutation motor control protocol 62 may provide generally forced rotor angle at motor startup. Operation at low speed can be less reliable with smoother operation at greater speed with lower current draw. Positional control of the working element 24 with the field oriented commutation motor control protocol 62 may be less reliable than the other methods.

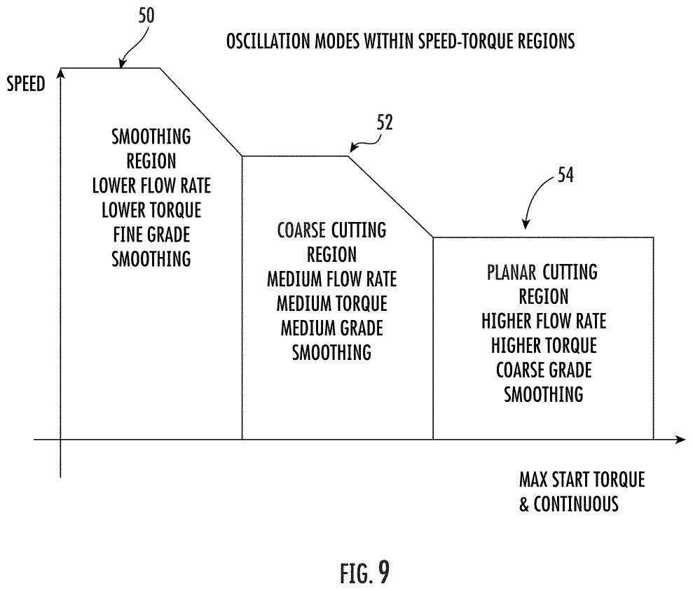

The system 10 is also configured such that when the system 10 implements different motor control protocols, the system 10 takes into consideration the factors affecting performance for different applications, different speed and torque ranges and other factors. For example, if a desired medical procedure requires a smoothing action, as shown in smoothing region 50 of FIG. 9, then the working element 24 may be operated at a high speed and lower torque with a low fluid flow rate. In the smoothing region 50, the working element 24 may be operated for polishing bone at a higher speed, especially when using a burr in the reverse direction, which is desirable because surgeons have more control over the burr and can polish bone. Surgeons often use faster speeds even in the forward direction because their perception that faster is better. In at least one embodiment, the high speed may be about 6200 revolutions per minute (RPM) for aggressive bone resection because such speed allows flutes on a working element 24 to have time and dig into the bone. Operating the working element 24 within the smoothing region 50 may be beneficial for tissue resection because such operation may enable many small bites to be taken in a short amount of time which may reduce clogging and increase resection rate. Procedures where polishing is of value is in operations on the hip where a surgeon typically wants to polish the femur where it articulates with the acetabulum, which is often referred to as a CAM resection. Operating the working element 24 within the smoothing region 50 can also be used in an acetabuloplasty prior to a labral repair. Some surgeons value having a polishing burr for prepping the glenoid in a shoulder prior to a labral repair and for prepping the tuberosity before a rotator cuff repair. Operating the system 10 within the smoothing region 50 of FIG. 9, whereby the working element 24 is operated at high speeds with lower torque, is most efficient when the motor control protocol employed is the Field Oriented Control (FOC) protocol 62. The Field Oriented Control (FOC) protocol 62 may maximize motor operational efficiency and reduce internal heating, unless the speed is increased to a point that would require switching into a sinusoidal protocol because the computational loading required to maintain the FOC protocol 62 would not be possible at such high speed.

An example situation in which the Field Oriented Control (FOC) protocol 62 may be the most efficient motor control protocol is a surgical procedure in which a surgeon is preparing a bone surface for a graft or general soft tissue resection. Polishing bone at a higher speed, especially when using a burr in the reverse direction, is desirable because surgeons have more control over the burr at higher speeds and can effectively polish bone. Surgical procedures using polishing include, but are not limited to: surgical procedures in the hip where a surgeon wants to polish the femur where it articulates with the acetabulum, which may be referred to as a CAM resection; an acetabuloplasty prior to a labral repair; preparation of a glenoid in a shoulder prior to a labral repair and preparation of a tuberosity before a rotator cuff repair. In at least one embodiment, the higher speed of the working element 24 may be a rotational speed of between about 5,000 RPM and 7,000 RPM, and in at least one embodiment, may be about 6,200 RPM for optimal, aggressive, bone resection. Such speed range for the working element 24 may be beneficial for tissue resection because such speed would enable the working element 24 to take many small bites in a short amount of time, which could reduce clogging and increase a resection rate.

For motor operation in the smoothing region 50 of FIG. 9, the Field Oriented Control (FOC) protocol 62 may be implemented by the controller 38 via the processor 32 in automatic or manual modes. In manual mode, a user may choose, via menu selections, the operational mode of the handheld device 14 such that the motor 12 operates in a high speed mode and low torque mode, and the processor 32 may then operate the motor 12 via the Field Oriented Control (FOC) protocol 62. A used may also input a type of tool attachment, such as a shaver, is attached to the system 10. A user may choose to operate the working element 24 in oscillation mode or continuous mode. In another embodiment, a user may input into the system 10 a type of surgical procedure to be performed, and the processor 32 may choose the Field Oriented Control protocol 62, high speed mode and low torque mode when the surgical procedure entered matches with a procedure associated with these operating modes in the memory 28.

Similarly, if a desired medical procedure requires coarse cutting, as shown in coarse cutting region 52 of FIG. 9, then the working element 24 may be operated at a medium speed, which may be less than the speed in smoothing region 50, and medium torque, which may be greater than the torque in the smoothing region 50, with a medium fluid flow rate, which may be greater than the fluid flow rate in the smoothing region 50. Operating the working element 24 at a medium speed within the cutting region 52 may be useful for bone and tissue removal. Operating the system 10 within the coarse cutting region 52 of FIG. 9, whereby the working element 24 is operated at medium speeds with medium torque, is most efficient when the motor control protocol employed is the Field Oriented Control (FOC) protocol 62. The Field Oriented Control (FOC) protocol 62 may be efficient in operating the motor at medium speeds with medium torque demands unless the torque demand is increased to a point that would require switching into a trapezoidal protocol 60 to improve cutting efficiency. An example situation in which the Field Oriented Control (FOC) protocol 62 may be the most efficient motor control protocol is a surgical procedure in which a surgeon is performing a surgical procedure in which the surgeon is conducting more aggressive bone or soft tissue resection. Operating the working element 24 in the middle of a range of speed and torque is desirable for bone and tissue removal.

For motor operation in the coarse cutting region 52 of FIG. 9, the Field Oriented Control (FOC) protocol 62 may be implemented by the controller 38 via the processor 32 in automatic or manual modes. In manual mode, a user may choose the operational mode of the handheld device 14 such that the motor 12 operates in a medium speed mode and medium torque mode, and the processor 32 may then operate the motor 12 via the Field Oriented Control (FOC) protocol 62. A user may choose to operate the working element 24 in oscillation mode or continuous mode. A used may also input a type of tool attachment, such as a shaver, is attached to the system 10. In another embodiment, a user may input into the system 10 a type of surgical procedure to be performed, and the processor 32 may choose the Field Oriented Control (FOC) protocol 62, high speed mode and low torque mode when the surgical procedure entered matches with a procedure associated with these operating modes in the memory 28.

If a desired medical procedure requires planar cutting, as shown in planar cutting region 54 of FIG. 9, then the working element 24 may be operated at a lower speed, which may be less than the speed in coarse cutting region 52, and higher torque, which may be greater than the torque in the coarse cutting region 52, with a higher fluid flow rate, which may be greater than the fluid flow rate in the coarse cutting region 52. Operating the system 10 within the planar cutting region 54 of FIG. 9, whereby the working element 24 is operated at lower speeds with higher torque, is most efficient when the motor control protocol employed is the Sinusoidal Motor Control protocol 64. The Sinusoidal Motor Control protocol 64 may be efficient in operating the motor at lower speeds with higher torque demands unless the torque demand is increased to a point that would require switching into a trapezoidal motor control protocol because of aggressive loading to prevent motor stalls. An example situation of using the Sinusoidal Motor Control protocol 64 might be if a surgeon is performing a surgical procedure requiring very aggressive bone resection, such as more aggressive bone resection than accomplished in the coarse cutting region 52, or meniscus resection. Use of the Sinusoidal Motor Control protocol 64 to create lower speeds with higher torque may best support driving a drill as the working element 24, whereby a drill, such as, but not limited to, a SHAVERDRILL, is used for reaming but could be useful when using a drill, such as, but not limited to, a SHAVERDRILL, to make pilot holes for implants before a labral repair or any product in which a drill is needed. The Sinusoidal Motor Control protocol 64 may be used to drive a working element 24, such as but not limited to, a microfracture instrument, such as, but not limited to, a POWERPICK, at lower speeds with higher torque for microfracture procedures to help create fibrocartilage in an OCD lesion in a femur, a glenoid, a patella, or an ankle. The Sinusoidal Motor Control protocol 64 may also be used to drive the working element 24, such as a drill, at lower speeds with higher torque, to promote healing in ACL repairs and in rotator cuff repairs by promoting bleeding.

For motor operation in the planar cutting region 54 of FIG. 9, the Sinusoidal Motor Control protocol 64 may be implemented by the controller 38 via the processor 32 in automatic or manual modes. In manual mode, a user may choose, via menu selections, the operational mode of the handheld device 14 such that the motor 12 operates in a low speed mode and high torque mode, and the processor 32 may then operate the motor 12 via Sinusoidal Motor Control protocol 64. A used may also input a type of tool attachment, such as a drill, is attached to the system 10. A user may choose to operate the working element 24 in oscillation mode or continuous mode. In another embodiment, a user may input into the system 10 a type of surgical procedure to be performed, and the processor 32 may choose the Sinusoidal Motor Control protocol 64, low speed mode and high torque mode when the surgical procedure entered matches with a procedure associated with these operating modes in the memory 28.

The system 10 may also be configured to perform health monitoring of the motor operation so that if there is a detectable failure in the handpiece operation, the processor 32 could continue to proceed without causing a stop in the workflow by switching motor control protocols to a protocol that does not need the failed sensor or other failed component of the handheld device 14. Such capability may be advantageous in surgical procedures where a sensor failure is detected and a surgeon concludes that it is more desirable to complete the procedure using what may be a suboptimal sensorless control method for that procedure, as opposed to causing a disruption in the operation. The system 10 could alert the user, such as a surgeon, of a detected component failure. The surgeon could then manually switch motor control protocols or determine to not use the handpiece 14 and replace it with another handpiece 14. Alternatively, the system 10 could automatically switch motor control protocols. The processor 32 may perform instructions to operate the motor 12 continuously upon detection of failure of a component of the system 10. In at least one embodiment, the processor 32 may be configured to execute instructions to perform operations to drive the motor 12 continuously upon detection of failure of a component of the system 10, whereby the component detected as having failed is one or both of the sensors 16, 18, one sensor 16 being to monitor the magnetic flux field of the motor and the other sensor 18 configured to configured to monitor current to the motor 12.

During use, a user, such as, but not limited to, a surgeon or other medical professional, may provide input to the system, such as, but not limited to, the tool type, type of procedure to be undertaken, operational mode, and the like. The processor 32 of the medical device system 10 may choose a motor control protocol to operate the motor 12. In at least one embodiment, the processor 32 may implement a six-step hall based motor commutation control protocol 60. With this protocol, speed may be maintained with a proportional, integral, derivative control loop that compares set speed to measured speed and adjusts motor current through a pulse width modulated (PWM) signal that is driving more or less voltage to the motor 12. As load increases on the motor, speed drops and additional current is required, which is supplied by a higher PWM duty cycle to the console voltages driving the three phases of the brushless direct current (BLDC) motor 12. As the motor 12 rotates, a plurality of hall sensors, such as, but not limited to, three hall sensors, located on the motor 12 sense changes in the motors magnetic flux field and signal the position of the motor shaft to the controller 38, which allows the processor 32 to accurately sequence the voltage drivers in a manner that keeps the motor 12 turning in a consistent direction. By reversing the sequence via motor commutation, the rotation can be changed from forward to reverse.

Throughout use of the system 10, the processor 32 monitors all feedback systems, including the sensors 16, 18, and any input from a user, and analyzes such input. If the processor 32 determines that a different motor control protocol used to operate the motor 12 would be more suitable for a given situation, the system 10 may automatically change the motor control protocol during use, thereby providing the motor with the most effective motor control protocol at all times. Such operation enhances the efficiency of the system 10 and improves performance to the surgeon.

The foregoing is provided for purposes of illustrating, explaining, and describing embodiments of the disclosed devices.

We claim:

1. A medical device system, comprising:
a handheld rotary medical device, comprising:
a motor,
an inner drive shaft coupled to the motor;
at least one sensor configured to monitor the motor;
an elongated, tubular, outer housing with at least a portion of the inner drive shaft positioned within the outer housing; and
a working element at a distal end of the inner drive shaft;
a memory that stores instructions;
a processor that executes the instructions to perform operations, the operations comprising:
controlling the driving of the motor and the inner drive shaft by monitoring the at least one sensor configured to monitor the motor; and
dynamically switching motor control protocols while the motor is operating based on input from the at least one sensor configured to monitor the motor.

2. The medical device system of claim 1, wherein the at least one sensor configured to monitor the motor is at least one sensor configured to monitor the magnetic flux field of the motor.

3. The medical device system of claim 2, further comprising at least one sensor configured to monitor current to the motor.

4. The medical device system of claim 3, wherein the processor performs operations based on instructions to dynamically switch motor control protocols while the motor is operating based on input from the at least one sensor configured to monitor the magnetic flux field of the motor and input from the at least one sensor configured to monitor current to the motor.

5. The medical device system of claim 1, wherein the at least one sensor configured to monitor the motor is at least one sensor configured to monitor current to the motor.

6. The medical device system of claim 1, wherein the processor that performs operations based on instructions to dynamically switch motor control protocols based on input is configured to perform operations to operate the motor via at least one of the following motor control protocols: Six-Step Commutation, Hall-Based Sinusoidal Commutation and Field Oriented Commutation.

7. The medical device system of claim 1, wherein the processor is configured to execute instructions to perform operations to drive the motor continuously upon detection of failure of a component of the system.

8. The medical device system of claim 7, wherein the processor is configured to execute instructions to perform operations to drive the motor continuously upon detection of failure of a component of the system, whereby the component detected as having failed is the at least one sensor.

9. The medical device system of claim 1, wherein dynamically switching motor control protocols while the motor is operating based on input from the at least one sensor configured to monitor the motor comprises dynamically switching motor control protocols with the processor in an automatic mode in which the processor selects a motor control protocol based on results from comparing calculated parameters against thresholds.

10. The medical device system of claim 9, wherein the thresholds are fixed.

11. The medical device system of claim 9, wherein the thresholds are a function of measured noise floors which the processor uses to calculate minimum allowable signal to noise thresholds.

12. The medical device system of claim 9, wherein the thresholds are dynamic commutation switch control factors.

13. The medical device system of claim 9, wherein the processor comparing calculated parameters against thresholds further comprises the processor receiving user input that the processor uses to calculate parameters.

14. The medical device system of claim 9, wherein comparing calculated parameters against thresholds further comprises the processor receiving control input that the processor uses to calculate parameters.

15. The medical device system of claim 1, wherein dynamically switching motor control protocols while the motor is operating based on input from the at least one sensor configured to monitor the motor comprises dynamically switching motor control protocols with the processor in a manual mode in which the processor operates based off of input from a user.

16. A medical device system, comprising:
a handheld rotary medical device, comprising:
a motor,
an inner drive shaft coupled to the motor;
at least one sensor configured to monitor the motor;
an elongated, tubular, outer housing with at least a portion of the inner drive shaft positioned within the outer housing; and
a working element at a distal end of the inner drive shaft;
a memory that stores instructions;
a processor that executes the instructions to perform operations, the operations comprising:
controlling the driving of the motor and the inner drive shaft by monitoring the at least one sensor configured to monitor the motor;
dynamically switching motor control protocols while the motor is operating based on input from the at least one sensor configured to monitor the motor;
wherein the processor automatically detects a presence of the at least one sensor configured to monitor the motor; and
wherein the at least one sensor is configured to sense a magnetic flux field of the motor.

17. The medical device system of claim 16, wherein the processor that performs operations based on instructions to dynamically switch motor control protocols is configured to perform operations to operate the motor via at least one of the following motor control protocols at different times: Six-Step Commutation, Hall-Based Sinusoidal Commutation and Field Oriented Commutation.

18. The medical device system of claim 16, wherein the processor is configured to execute instructions to perform operations to drive the motor continuously upon detection of failure of a component of the system.

19. The medical device system of claim 16, wherein dynamically switching motor control protocols while the motor is operating based on input from the at least one sensor configured to monitor the motor comprises dynamically switching motor control protocols with the processor in an automatic mode in which the processor selects a motor control protocol based on results from comparing calculated parameters against thresholds.

20. A medical device system, comprising:
a handheld rotary medical device, comprising:
a motor,
an inner drive shaft coupled to the motor;
at least one sensor configured to monitor the motor;
an elongated, tubular, outer housing with at least a portion of the inner drive shaft positioned within the outer housing; and
a working element at a distal end of the inner drive shaft;
a memory that stores instructions;
a processor that executes the instructions to perform operations, the operations comprising:
controlling the driving of the motor and the inner drive shaft by monitoring the at least one sensor configured to monitor the motor;
dynamically switching motor control protocols while the motor is operating based on input from the at least one sensor configured to monitor the motor;
wherein the processor is configured to execute instructions to perform operations to drive the motor continuously upon detection of failure of a component of the system; and
wherein the at least one sensor is configured to sense a magnetic flux field of the motor via at least one hall sensor.
